(19) United States
(12) Patent Application Publication
(10) Pub. No.: US 2022/0257500 A1
(43) Pub. Date: Aug. 18, 2022

(54) USE OF A HYDRO-ALCOHOLIC EXTRACT OF EVENING PRIMROSE FOR HYDRATING SKIN AND INCREASING BARRIER FUNCTION

(71) Applicant: L'OREAL, Paris (FR)

(72) Inventors: Franck JUCHAUX, Aulnay-Sous-Bois (FR); Saliou NGOM, Aulnay-Sous-Bois (FR)

(21) Appl. No.: 17/621,858

(22) PCT Filed: Jun. 26, 2020
(86) PCT No.: PCT/EP2020/068120

§ 371 Date: Dec. 22, 2021

(30) Foreign Application Priority Data
Jun. 28, 2019 (FR) FR1907157

(51) Int. Cl. A61K 8/9789; A61Q 19/00

(52) U.S. Cl. CPC A61K 8/9789; A61Q 19/007

(57) ABSTRACT

The present invention relates to a hydro-alcoholic extract of above-ground parts of evening primrose (Oenothera biennis), to a composition containing the same, and to cosmetic uses thereof.

The present invention concerns the use of a hydro-alcoholic extract of evening primrose for improving barrier function, and for hydrating skin.

The skin is a tissue having contiguous cells anchored to each other. Skin tissue forms an outermost covering comprising sebaceous or sweat glands, and hair follicles. Skin and in particular the scalp form constantly renewed epithelia. Renewal, or desquamation, is a finely regulated, coordinated process leading to elimination of surface cells in a manner that is invisible and imperceptible.

Human skin is composed of two compartments, namely a surface compartment i.e. the epidermis, and a deep compartment i.e. the dermis.

The epidermis is conventionally divided into a basal layer of keratinocytes forming the germinal layer of the epidermis, a so-called spinous layer composed of several layers of polyhedral cells arranged on the germinal layers, one to three so-called granular layers composed of flattened cells containing separate cytoplasmic inclusions i.e. granules of keratohyalin, and finally a set of upper layers called cornified layers (or stratum corneum) composed of keratinocytes in the terminal stage of differentiation called corneocytes.

Corneocytes are anucleate cells chiefly composed of fibrous material containing cytokeratins, surrounded by a cornified envelope. New keratocytes are permanently produced to offset the continuous loss of epidermal cells at the cornified layer following a mechanism known as desquamation.

However, imbalance between the production of cells at the basal layer and the rate of desquamation can particularly lead to scaling on the skin surface. Similarly, a deficiency in terminal differentiation of the cells of the stratum corneum, for various reasons, can lead to the formation of thick cell clusters of large size and visible to the naked eye called «squamae», or in other situations to thinning of the stratum corneum. This can lead to weakening of the barrier properties of the epidermis, to chronic dehydration of the stratum corneum, loss of mechanical elasticity, skin tightness and to loss of skin radiance or transparency.

As examples of factors promoting this decrease in the quality of the skin surface, mention can be made of stress, winter climate, excess sebum or lack of hydration.

Weakening of the skin barrier can occur in the presence of external harsh factors such as irritants (detergents, acids, bases, oxidants, reducers, concentrated solvents, harmful gases or smoke) heat or weather extremes (cold, dryness, radiation), xenobiotics (undesirable allergenic micro-organisms) or internal harsh factors such as psychological stress.

One of the critical steps in the process of terminal differentiation of the stratum corneum is crosslinking of the protein precursors of the cornified envelope (CE). This phenomenon plays an essential role in the development and maintaining of skin cohesion, in the physical properties of the skin as barrier function, and is a crucial step in the process of terminal differentiation. The cornified envelope is an essential component of corneocytes.

Hydrating active substances conventionally used such as humectants, hydrating polymers or fats such as Vaseline, afford transient modification of the surface properties of the skin. These active substances can induce mechanical softening of the stratum corneum, an increase in the state of hydration thereof and/or improvement in skin microrelief through the formation of a film on the skin surface. In general, these effects do not persist over time and only last a few hours. In addition, after cleansing the skin, these active substances are removed and the effects of mechanical softening of the skin, improved texture or optical properties of the skin are caused to disappear.

Finally, dry skin which lacks radiance is often treated with hydrating active substances which have an effect on differentiation and maturation of the stratum corneum.

There is a need for active substances enabling the skin and in particular dry skin to maintain its barrier function.

There is therefore a need for active substances which improve the condition of the skin surface, in particular of dry skin, preventing feelings of tightness and discomfort in users; there is additional need for active substances which also improve the properties of skin texture and radiance.

The maintaining or restoring of a cornified envelope having proper maturation is essential for the preserving of a barrier function of good quality ensuring protection against harmful external factors and sustained skin hydration in particular of the epidermis.

The present invention allows these needs to be met. The inventors have now discovered that a specific hydro-alcoholic extract of above-ground parts (also called aerial parts) of evening primrose (Oenothera biennis) allows satisfactory hydration of the skin but also maintaining of the barrier function properties of the skin.

As shown in the examples, this extract is specific and has unique properties: unlike other extracts of the same plant, it allows an improvement in the barrier function and in skin hydration. This extract induces the expression of markers involved in the forming of the cornified layer and barrier function such as the markers CNFN (cornifelin), FLG2 (filaggrin 2), LCE3D («late cornified envelope 3D») and SPRR1A («small proline-rich protein 1A»). It is therefore of interest for improvement in barrier function and hydration. In addition, and surprisingly, the biological efficacy observed with this extract is not carried by one of these main components.

The present invention therefore relates to a hydro-alcoholic extract of above-ground parts (also called aerial parts) of evening primrose (Oenothera biennis).

The present invention also relates to the use of said extract to strengthen the barrier function and/or to improve skin hydration.

Typically, by strengthening the barrier function, said extract also improves the quality of the skin and/or radiance of complexion.

In addition, the extract allows the preventing and/or treating of cosmetic signs associated with dry skin.

The cosmetic signs associated with dry skin are particularly selected from among feelings of skin tightness and discomfort.

By «skin», it is meant the entire skin of the body and preferably the skin of the face, neck, neckline, arms and forearms, even further preferably facial skin (in particular the forehead, nose, cheeks, chin), and skin of the neck and neckline.

By «to prevent» or «prevention» in the invention it is meant the fact of reducing the probability of onset or risk of signs of the concerned phenomenon.

With the present invention it is therefore possible to impart beneficial properties to the skin in particular in sustainable manner, and particularly:
efficient barrier function;
hydrating effect;
elasticity and smooth texture of the skin;
surface morphology with scarce roughness, good tissue cohesion and improved visual appearance of the skin.

An increase in skin dryness is often observed with age; however, such conditions of skin dryness can also occur in younger subjects. Skin dryness is a physiological condition which can be present in young persons without any pathological cause. This dryness can be constitutional as is often the case in persons with pale, thin and/or fragile skin.

Also, numerous external factors can lead to drying of the skin or to aggravating the condition of an already dry skin. Among these factors, mention can be made of difficult weather conditions, sun rays, exposure to some chemical or therapeutic agents.

At physiological level, dry skin is often associated with a decrease in skin hydration rate and impaired barrier function, measured by imperceptible loss of water. At sensory level, it is notably characterized by feelings of skin tightness and/or tautness. For obvious reasons, these signs are a source of discomfort and even pain.

Yet, in some situations whether unbalanced food diet, repeated external physical or chemical attack (e.g. UV radiation, pollution, wind, cold or air-conditioning) or psychological factors (fatigue, stress), the human epidermis can display qualitative or quantitative changes in the composition and/or lipid synthesis thereof.

The extract of the invention therefore proves to be particularly efficient for:
treating conditions of skin dryness;
treating dry skin;
treating itching and/or tightness associated with dry skin;
physiologically restoring a suitable state of hydration of the stratum corneum;
treating hypo-seborrheic dry skin;
improving the comfort of dry skin; or
combating a dull and/or lacklustre appearance of the skin as a result of dryness.

In particular, the extract of the invention is useful as agent for improving skin hydration. In one embodiment of the invention, the extract of the invention is intended to prevent and/or treat dry skin and/or to reduce the signs associated with skin dryness whether constitutional or acquired.

The invention also relates to a composition, preferably cosmetic, comprising a hydro-alcoholic extract of above-ground parts of evening primrose (Oenothera biennis) in a cosmetically acceptable medium.

A further subject of the present invention is a non-therapeutic cosmetic method for care of keratin material such as skin, comprising the topical application to this keratin material of at least an hydro-alcoholic extract of above-ground parts of evening primrose (Oenothera biennis) such as described above, or of a composition such as defined above. More particularly, a subject of the invention is a cosmetic treatment method such as defined above to improve and/or strengthen the barrier function of the skin, in particular to promote the maintaining of skin having a barrier function of good quality.

The invention first concerns a hydro-alcoholic extract of above-ground parts of evening primrose (Oenothera biennis).

The plant used in the invention can be Oenothera biennis ssp maritima.

By «above-ground parts» or “aerial parts”, it is meant the flowers, leaves and/or stems. In particular, the above-ground parts do not comprise the roots or rhizomes.

Plant extraction is a process intended to extract some constituents contained in plants. It is a solid/liquid separation operation: a solid body (the plant and here the above-ground parts of evening primrose) is contacted with a liquid (the solvent, here a mixture of water and at least one alcohol). The compounds of interest are solubilised and contained in the solvent. The solution obtained corresponds to the desired extract.

The solvent can then be removed to isolated the plant extract: when it is fully removed a dry extract is obtained.

Therefore, by «dry extract» it is meant an extract obtained after removing the extraction solvent. Preferably, the dry extract comprises less than 10 weight %, preferably less than 5 weight %, preferably less than 3 weight %, preferably less than 1 weight %, preferably less than 0.5 weight % of extraction solvent (here an alcohol or mixture of water and at least one alcohol) relative to the total weight of the extract. Preferably, the dry extract is free of extraction solvent.

Preferably, in a first embodiment, the extract of the invention is a dry extract.

If the solvent is not fully removed, the term fluid extract is used. Preferably, the fluid extract has a content of extraction solvent (here mixture of water and at least one alcohol) higher than 10 weight % relative to the total weight of extract.

Preferably, in a second embodiment, the extract of the invention is a fluid extract.

By «hydro-alcoholic extract», it is meant an extract obtained with a solvent which is a mixture of water and at least one alcohol.

In particular, the alcohol is a monoalcohol having 2 to 6 carbon atoms. Preferably, the alcohol is ethanol or methanol, preferably ethanol.

Preferably, the mixture of water and at least one alcohol is a mixture comprising from 20 to 60 volume % of water relative to the total volume of the mixture, and 40 to 80 volume % of alcohol relative to the total volume of the mixture. Preferably, the mixture of water and at least one alcohol is a mixture comprising from 30 to 55 volume %, preferably 45 to 55 volume % of water relative to the total volume of the mixture, and from 45 to 60 volume %, preferably 47 to 55 volume % of alcohol relative to the total volume of the mixture. Preferably, the mixture is a mixture of water and a single alcohol.

More preferably, the mixture of water and at least one alcohol is a mixture comprising 50 volume % of water relative to the total volume of the mixture, and 50 volume % of alcohol, preferably ethanol, relative to the total volume of the mixture (50% mixture).

Preferably, the hydro-alcoholic extract of above-ground parts of evening primrose according to the invention is obtained from the above-ground parts that are previously ground and extracted with a hydro-alcoholic mixture (i.e. mixture of water and at least one alcohol having 2 to 6 carbon atoms, preferably ethanol), preferably a 50% mixture.

The genus Oenothera belongs to the Onagraceae family which comprises about 145 species, growing in regions of North and South America and in Europe. Some species, in particular Oenothera biennis (or biannual evening primrose) are the source of evening primrose oil derived from the seeds which contains a high percentage of unsaturated fatty acids and in particular gamma-linoleic acid. The defatted seeds of evening primrose form waste from pharmaceutical and cosmetic industries and are intensively researched for their high content of flavonoids and tannins.

Antioxidant, anti-inflammatory and anti-tumoral effects have been attributed to the methanolic extract of defatted seeds. On the other hand, the above-ground parts often form waste from the evening primrose oil supply chain obtained from seeds, and are rarely recovered for reuse. Research on the chemical composition and properties of extracts prepared from the above-ground parts of species of Oenothera are fairly limited and show that the above-ground parts contain flavonoids, phenolic acids, tannins and triterpenes. Oenothera biennis that is used is a halophyte, i.e. capable of developing in an environment subjected to saline stress.

In the invention, the biomass used is composed of the dried, above-ground parts of evening primrose (Oenothera biennis). In particular, the variety Oenothera biennis ssp maritima can be used. Preferably, this plant is cultivated in the North Brittany region (France) by Setalg. The above-ground parts are preferably harvested when in full bloom in July.

The hydro-alcoholic extract of above-ground parts of evening primrose (Oenothera biennis) according to the invention is obtained with a method comprising extraction of the above-ground parts, preferably previously ground, in a mixture of water and at least one alcohol, preferably a monoalcohol having 2 to 6 carbon atoms, preferably ethanol. Preferably, said extraction is performed at ambient temperature (i.e. around 20-25° C.).

The hydro-alcoholic extract of the invention is preferably obtained as described in Example 1. In particular, it is obtained with the method comprising the following steps:

a): grinding the dried, above-ground parts of evening primrose to fine particles in a grinder (particle size: 1-2 mm);

b): extracting, via maceration in a mixture of water and at least one alcohol at a temperature of 15° C. to 45° C., in particular 15° C. to 40° C. and particularly at ambient temperature, in particular by adding 10 g to 500 g of plant powder obtained at a) per litre of water and alcohol mixture, in particular 50 g to 200 g and more particularly 70 g to 150 g, e.g. 100 g of plant powder obtained at a) per litre of water and alcohol mixture, and holding this temperature for 1 to 5 hours, in particular 1 to 3 h, more particularly 1 h30 to 2 h30 e.g. 2 hours. Preferably, the mixture comprises from 20 to 60 volume %, preferably 30 to 55 volume %, more preferably 45 to 55 volume %, further preferably 50 volume of water relative to the total volume of the mixture, and from 40 to 80 volume %, preferably 45 to 60 volume %, more preferably 47 to 55 volume %, further preferably 50 volume of alcohol, preferably ethanol, relative to the total volume of the mixture; and

c): filtering the mixture obtained at b), in particular on a Whatman GF/C filter having porosity of 1.2 μm;

d): partially or fully evaporating the alcohol from the filtered mixture obtained at c), typically in a rotary evaporator at 35° C.; and

e): optionally freeze-drying the aqueous residue obtained at d), typically for 24 hours.

If step d) is full evaporation of the alcohol followed by the freeze-drying step e), the extract is obtained in dry form (dry extract) which can optionally be ground and/or packaged typically in a glass bottle.

The hydro-alcoholic dry extract of the invention is preferably in the form of a powder 1.5 c)/0 soluble in water, 5% soluble in a 50:50 water/ethanol mixture, and insoluble in pure ethanol. It is preferably light brown in colour.

Preferably, the hydro-alcoholic extract of above-ground parts of evening primrose according to the invention is obtained from the above-ground parts that are previously ground and extracted with a hydro-alcoholic mixture (i.e. mixture of water and at least one alcohol having 2 to 6 carbon atoms, preferably ethanol), preferably a 50% mixture (in volume), preferably in a ratio of around 100 g of above-ground parts powder for about 1 L of hydro-alcoholic mixture, at ambient temperature; then filtered with a filter having porosity of 0.8 to 2 μm; evaporated and optionally freeze-dried.

Preferably, the hydro-alcoholic extract of above-ground parts of evening primrose according to the invention is obtained from the above-ground parts that are previously ground and extracted with a 50:50 water/ethanol mixture (in volume), preferably in a ratio of around 100 g of above-ground parts powder for about 1 L of water/ethanol mixture, at ambient temperature; then filtered with a filter having porosity of 0.8 to 2 μm, preferably of around 1.2 μm; evaporated and optionally freeze-dried.

Preferably, the hydro-alcoholic extract of the invention comprises at least two, preferably at least three, preferably at least four, preferably at least five molecules selected from among oenothein B, quercetin, ellagic acid, hyperoside, isoquercitrin (or isoquercetin) and luteoline-7-O-glucuronide.

Preferably, the hydroalcoholic extract of the invention comprises the six following molecules: oenothein B, quercetin, ellagic acid, hyperoside, isoquercitrin and luteoline-7-O-glucuronide.

The invention also concerns a composition, preferably cosmetic composition, comprising in a cosmetically acceptable medium a hydro-alcoholic extract of above-ground parts of evening primrose (Oenothera biennis) according to the invention.

Preferably, the extract of the invention is prepared by mixing between 20 g and 500 g, preferably between 50 g and 250 g, of powder of the plant above-ground parts per 1 L of water and alcohol (solvent) mixture, preferably with 100 g of powder of the plant above-ground parts per 1 L of water and alcohol (solvent) mixture.

More particularly, when the extract is in the form of a dry extract, said dry extract is contained in an amount ranging from 0.0001 to 10 weight %, preferably from 0.001 to 5 weight %, relative to the total weight of the composition.

More particularly, when the extract is in the form of an extract in a hydro-alcoholic medium, said extract is contained in an amount ranging from 0.0001% to 30 weight %, preferably 0.001% to 25 weight % relative to the total weight of the composition.

By «cosmetically acceptable medium», it is meant a medium compatible with the skin, mucosa and/or appendages.

The compositions of the invention can be in any galenic form conventionally used for topical application, and in particular in the form of aqueous or hydroalcoholic solutions, oil-in-water (O/W) or water-in-oil (W/O) or multiple (triple: W/O/W or O/W/O) emulsions, aqueous gels, or dispersions of a fatty phase in an aqueous phase using spherules, these spherules possibly being lipid vesicles of ionic and/or nonionic type (liposomes, niosomes, oleosomes). These compositions are prepared following usual methods.

The compositions of the invention can also be in anhydrous form e.g. in oil form. By «anhydrous composition» it is meant a composition containing less than 1 weight % of water, even less than 0.5 weight % of water, and in particular it is free of water, there being no addition of water when preparing the composition but corresponding to residual water afforded by the mixed ingredients. Advantageously, the compositions of the invention are in the form of a gel or emulsion, powder or paste.

In addition, the composition of the invention can be more or less fluid and have the appearance of a white or coloured cream, ointment, milk, lotion, serum, paste, foaming gel, a care product, astringent or foam. It can also optionally be applied to the skin in aerosol form. And it can be in solid form e.g. in stick form.

When the composition used in the invention comprises an oily phase, the latter preferably contains at least one oil. It may also contain other fats.

As oils that can be used in the composition of the invention, mention can be made for example of:
hydrocarbon oils of animal origin such as perhydrosqualene;
hydrocarbon oils of vegetable origin such as liquid triglycerides of fatty acids having 4 to 10 carbon atoms e.g. the triglycerides of heptanoic or octanoic acids, or oils for example such as sunflower seed, corn, soy, pumpkin, grapeseed, sesame, hazelnut, apricot, macadamia, arara, sunflower seed, castor, avocado, the triglycerides of caprylic/capric acids such as those marketed by Stearineries Dubois or those sold under the tradenames Miglyol 810, 812 and 818 by CREMER OLEO, jojoba oil, shea butter oil;
synthetic esters and ethers, in particular of fatty acids e.g. oils of formulas RCOOR2 and ROR2 where R is the remainder of a fatty acid having 8 to 29 carbon atoms, and R2 is a hydrocarbon chain whether or not branched having 3 to 30 carbon atoms e.g. Purcellin oil, isononyl isononanoate, isopropyl myristate, ethyl-2-hexyl palmitate, octyl-2-dodecyl stearate, octyl-2-dodecyl erucate, isostearyl isostearate; hydroxyl esters such as isostearyl lactate, octyl hydroxystearate, octyldodecyl hydroxystearate, diisostearyl-malate, triisocetyl citrate, heptanoates, octanoates, decanoates of fatty alcohols; polyol esters such as propylene glycol dioctanoate, neopentyl glycol diheptanoate and diethylene glycol diisononanoate; and the esters of pentaerythritol such as pentaerythrityl tetraisostearate;
linear or branched hydrocarbons of mineral or synthetic origin such as paraffin oils whether or not volatile, and derivatives thereof, Vaseline, polydecenes, hydrogenated polyisobutene such as Parleam® oil;
fatty alcohols having 8 to 26 carbon atoms such as cetyl alcohol, stearyl alcohol and mixture thereof (cetylstearyl alcohol), octyldodecanol, 2-butyloctanol, 2-hexyldecanol, 2-undecylpentadecanol, oleic alcohol or linoleic alcohol;
partially hydrocarbon- and/or silicone-containing fluorinated oils such as those described in document JP-A-2-295912;
silicone oils such as polydimethylsiloxanes (PDMS) whether or not volatile with linear or cyclic silicone chain, liquid or pasty at ambient temperature, in particular cyclopolydimethylsiloxanes (cyclomethicones) such as cyclohexasiloxane; polydimethylsiloxanes comprising alkyl, alkoxy or phenyl groups, either pendant or at the end of the silicone chain, groups having 2 to 24 carbon atoms; phenylated silicones such as phenyltrimethicones, phenyldimethicones, phenyltrimethylsiloxydiphenyl-siloxanes, diphenyldimethicones, diphenylmethyldiphenyl trisiloxanes, 2-phenylethyltrimethyl-siloxysilicates, an polymethylphenylsiloxanes;
and mixtures thereof.

By hydrocarbon-containing oil in the above list of oils, it is meant any oil mostly comprising atoms of carbon and hydrogen, and optionally ester, ether, fluorinated groups, carboxylic acid and/or alcohol.

The other fats possibly contained in the oily phase are fatty acids for example having 8 to 30 carbon atoms, such as stearic acid, lauric acid, palmitic acid and oleic acid; waxes such as lanoline, beeswax, Carnauba or Candelilla wax, paraffin or lignite waxes, or microcrystalline waxes, ceresin or ozokerite, synthetic waxes such as polyethylene waxes, Fischer-Tropsch waxes; silicone resins such as trifluoromethyl-C1-4-alkyldimethicone and trifluoropropyldimethicone; and silicone elastomers such as the products marketed under the trade names KSG by Shin-Etsu, under the trade names “Trefil”, “BY29” or “EPSX” by Dow Corning or under the trade names “Gransil” by Grant Industries.

These fats can be selected in various manners by persons skilled in the art to prepare a composition having the desired properties of consistence or texture for example.

The composition can comprise at least one emulsifier, selected in particular from among amphoteric, anionic, cationic or nonionic emulsifiers, used alone or in a mixture, and optionally a co-emulsifier. The emulsifiers are selected in a manner suitable to obtain the desired emulsion (water-in-oil or oil-in-water). The emulsifier and co-emulsifier are generally contained in the composition in a proportion ranging from 0.3 to 30 weight %, preferably 0.5 to 20 weight % relative to the total weight of the composition.

The composition of the invention may also contain some usual ingredients in the cosmetic field, such as hydrophilic or lipophilic gelling agents, preserving agents, perfumes, fillers, pasty fats, sun protection or UV filters, odour absorbers, colouring agents, bases, acids, sequestering agents, polyols, nonionic, anionic or cationic surfactants.

The composition of the invention may evidently comprise other barrier effect active substances and/or other hydrating active substances differing from the extract of the invention.

The amounts of these different active substances are those conventionally used in the field under consideration, for example from 0.01 to 20 weight % of the total weight of the composition. These active substances, depending on type, can be added to the fatty phase, aqueous phase and/or in lipid vesicles.

The compositions of the invention may further comprise at least one aqueous phase. The aqueous phase contains water and optionally other organic water-soluble or water-miscible solvents. One aqueous phase suitable for the invention may comprise water selected for example from among natural spring water e.g. La Roche-Posay, Vittel, or Vichy, or a floral water.

Evidently, persons skilled in the art will take care to select any compounds to be added to the composition of the invention, and the amounts thereof, in such manner that the advantageous properties intrinsically attached to the composition conforming to the invention are not or are not substantially impaired by the envisaged addition.

As previously indicated, the present invention also relates to the use of a hydro-alcoholic extract of above-ground parts of evening primrose (Oenothera biennis) to strengthen the barrier function and/improve hydration of the skin.

A further subject of the present invention is a non-therapeutic cosmetic method for care of keratin material such as the skin, comprising the topical application to this keratin material of at least an hydro-alcoholic extract of above-ground parts of evening primrose (Oenothera biennis) such as described above, or of a composition such as defined above. More particularly, the subject of the invention is a cosmetic treatment method such as defined above to improve and/or strengthen the barrier function of the skin, in particular to promote the maintaining of skin having a barrier function of good quality.

The method of the invention may comprise repeated application e.g. 1 to 3 times daily or over several days, preferably 1 to 2 times daily, and in particular over an extended time of at least 4, even 4 to 15 weeks, possibly with one or more periods of interruption. In one embodiment, the cosmetic treatment method of the invention may comprise a single application. Preferably, a method of the invention comprises the topical application of a composition of the invention to the skin, in particular to facial skin.

Practical examples illustrating the invention are now given that are in no way limiting. In the examples, the temperature is ambient temperature (20° C.) and expressed in degrees Celsius unless otherwise stated, and the pressure is atmospheric pressure unless otherwise stated.

### EXAMPLE 1: PREPARATION OF THE HYDRO-ALCOHOLIC EXTRACT OF ABOVE-GROUND PARTS OF EVENING PRIMROSE (OENOTHERA BIENNIS) ACCORDING TO THE INVENTION

The extract of the invention was prepared with the following method:
Grinding to fine particles the dried, above-ground parts of evening primrose in a grinder (particle size: 1-2 mm). The above-ground parts are the parts exposed to open air (leaves, stems and flowers) under normal growth conditions of evening primrose, which in particular excludes the roots;
Extracting, via maceration in an ethanol/water mixture (50:50 by volume, i.e. v/v) at ambient temperature, by adding 100 g of plant powder per litre of solvent, and holding at this temperature for 2 hours;
Followed by filtering on a Whatman GF/C filter of pore size 1.2 μm;
Evaporating the alcohol in a rotary evaporator at 35° C.;
Freeze-drying the aqueous residue for 24 hours;
Grinding and packaging the dry extract obtained in a glass bottle.

A dry, hydro-alcoholic powder extract is obtained of above-ground parts of evening primrose.

Additional crude extracts were prepared by replacing the ethanol/water mixture (50:50 v/v) by:
solely water, allowing an aqueous dry extract to be obtained of above-ground parts of evening primrose (hereafter: «comparative aqueous extract»);
solely ethanol, allowing an ethanolic dry extract to be obtained of above-ground parts of evening primrose (hereafter: «comparative ethanolic extract»); or
solely ethyl acetate, allowing an ethyl acetate dry extract to be obtained of above-ground parts of evening primrose (hereafter: «comparative ethyl acetate extract»).

A further additional crude extract was prepared by macerating 200 g of powder of above-ground parts of evening primrose in 1 L of water (i.e. a higher ratio: 20:100) for 2 h at ambient temperature, followed by filtering through 100 μm (i.e. different filtration), evaporation in a rotary evaporator and freeze-drying. The extract obtained is called «comparative aqueous extract 2».

### EXAMPLE 2: EVALUATION OF EFFICACY ON HYDRATION AND MODULATION OF THE BARRIER FUNCTION

Principle:

The purpose of this study was to evaluate the effects of the hydro-alcoholic extract of above-ground parts of evening primrose according to the invention, such as described in Example 1, on the expression of transcripts (mRNA) involved in the barrier function and skin hydration, by RT-qPCR.

The hydro-alcoholic extract of above-ground parts of evening primrose of the invention obtained according to Example 1 was evaluated at 0.1 mg/mL.

The different markers examined were: CNFN, FLG2, LCE3D and SPRR1A.

Protocol (Experimental Conditions):

Normal, human epidermal keratinocytes were used to conduct the study.

Cytotoxicity

The normal, human epidermal keratinocytes were seeded in 96-well culture plates and cultured at 37° C. under 5% CO2 in a culture medium for 24 hours. The culture medium was then replaced by a culture medium containing or not containing (control) the compounds to be tested (8 concentrations tested) and the cells were incubated for 24 hours. All conditions were performed with n=2. After incubation, cell viability was measured using a standard test to measure mitochondrial activity with Alamar Blue®.

RT-qPCR Analysis of Gene Expression

Normal, human epidermal keratinocytes were seeded in 48-well culture plates and cultured in culture medium for 3 days at 37° C. under 5% CO2 with renewal of the culture medium after the first 24 hours of culture. After incubation, the culture medium was replaced by test medium (supplemented with 1.5 mM CaCl2) containing or not containing (control) the compounds to be tested, and the cells were incubated for 24 hours. All conditions were performed with n=2.

After treatment, the culture media were removed and the cells rinsed twice with PBS (w/o CaCl2, w/o MgCl2). Total RNAs were isolated using a magnetic bead extraction kit, following the manufacturer's instructions (MagMAX™-96 Total RNA Isolation Kit, Ambion). Quantification of RNAs and quality control thereof were analysed using Labchip GX (Perkin Elmer).

Expression of the selected transcripts was analysed by two-step quantitative PCR. First, the cDNAs were retro-transcribed from the RNAs using the Quantitect® Reverse transcription kit (QIAGEN) following the manufacturer's instructions. The quantitative PCR experiments were performed using a LightCycler® 480 Real-Time PCR System in 384-well plate (Roche) following the incorporation technique of SYBR®Green (Roche) (Table 4).

Results and Conclusions:

The results are expressed as fold change (Fc) as compared with the non-treated control, after normalising relative expressions in relation to expression of a housekeeping gene (GAPDH). The expression of a gene is considered to be stimulated when it is multiplied at least by 1.5.

Table 1 shows the effect of a hydro-alcoholic extract of above-ground parts of evening primrose according to the invention on the expression of transcripts (mRNA) involved in the barrier function.

TABLE 1

| | CNFN | FLG2 | LCE3D | SPRR1A |
|---|---|---|---|---|
| | Fold change vs Control (Fc) | | | |
| Control | 1 | 1 | 1 | 1 |
| Hydro-alcoholic extract of the | 44.09 | 3.73 | 49.06 | 8.09 |
| invention at 0.1 mg/mL | | | | |

The results show that the hydro-alcoholic extract of above-ground parts of evening primrose according to the invention, at 0.1 mg/mL, induces the expression of markers involved in forming of the cornified layer and barrier function (Eve Toulza, Nicolas R Mattiuzzo, Marie-Florence Galliano, Nathalie Jonca, Carole Dossat, Daniel Jacob, Antoine de Daruvar, Patrick Wincker, Guy Serre, Marina et al. Large-scale identification of human genes implicated in epidermal barrier function. Genome Biology 8, R107 (2007)) (CNFN, FLG2, LCE3D, SPRR1A).

The hydro-alcoholic extract of evening primrose according to the invention is therefore of interest for skin ageing by improving the barrier function and hydration (R. K. Chaudhuri, K. Bojanowski et al. Improvement of hydration and epidermal barrier function in human skin by a novel compound isosorbide dicaprylate. International Journal of Cosmetic Science 39, 518-526 (2017)).

Table 2 compares the effects of the comparative aqueous extract of evening primrose and of the hydro-alcoholic extract of evening primrose according to the invention on the expression of transcripts (mRNA) involved in the barrier function.

TABLE 2

| | CNFN | FLG2 | LCE3D | SPRR1A |
|---|---|---|---|---|
| | Fold change vs Control (Fc) | | | |
| Control | 1 | 1 | 1 | 1 |
| Hydro-alcoholic extract of the | 44.09 | 3.73 | 49.06 | 8.09 |
| invention at 0.1 mg/mL | | | | |
| Comparative aqueous extract 2 at | 7.77 | 3.62 | 5.18 | 2.5 |
| 0.1 mg/mL | | | | |

The results show that the two evening primrose extracts induce the expression of markers involved in forming of the cornified layer and barrier function (CNFN, FLG2, LCE3D and SPRR1A). However, the effects observed with the hydro-alcoholic extract of the invention are 3 to 9 times greater than those observed with the comparative aqueous extract except with regard to FLG2 for which equivalent stimulations were observed.

The hydro-alcoholic extract of evening primrose according to the invention is therefore of interest for skin ageing by improving the barrier function and hydration.

Table 3 compares the effects of a hydro-alcoholic extract of above-ground parts of evening primrose according to the invention with those of the main described constituents thereof on the expression of transcripts (mRNA) involved in barrier function and hydration.

TABLE 3

| | CNFN | FLG2 | LCE3D | SPRR1A |
|---|---|---|---|---|
| | Fold change vs Control (Fc) | | | |
| Control | 1 | 1 | 1 | 1 |
| Hydro-alcoholic extract of the | 44.09 | 3.73 | 49.06 | 8.09 |
| invention at 0.1 mg/mL | | | | |
| Ellagic acid at 0.004 mg/mL | 0.64 | 1.22 | 0.51 | 0.96 |
| Quercetin at 0.1 mg/mL | 0.13 | 0.12 | 0.2 | 0.13 |
| Oenothein B at 0.004 mg/mL | 0.76 | 1.36 | 0.87 | 1.07 |
| Hyperoside at 0.1 mg/mL | 0.61 | 0.5 | 0.61 | 0.64 |
| Isoquercitrin at 0.1 mg/mL | 0.54 | 0.9 | 0.47 | 0.57 |
| Luteoline-7-O-glucuronide at | 0.42 | 0.4 | 0.44 | 0.41 |
| 0.1 mg/mL | | | | |

These main described components are oenothein B, quercetin, ellagic acid, hyperoside, isoquercitrin and luteoline-7-O-glucuronide.

The results show that the biological efficacy observed is not carried by one of these main components of the hydro-alcoholic extract of above-ground parts of evening primrose. These components even exhibit an opposite effect to the effect observed with the extract.

Table 4 gives the effects of different types of extracts of above-ground parts of evening primrose on the expression of transcripts (mRNA) involved in the barrier function.

TABLE 4

| | CNFN | FLG2 | LCE3D | SPRR1A |
|---|---|---|---|---|
| | Fold change vs Control (Fc) | | | |
| Control | 1 | 1 | 1 | 1 |
| Hydro-alcoholic extract of the | 44.09 | 3.73 | 49.06 | 8.09 |
| invention at 0.1 mg/mL | | | | |
| Comparative aqueous extract 2 | 7.77 | 3.62 | 5.18 | 2.5 |
| at 0.1 mg/mL | | | | |
| Comparative ethyl acetate | 3.64 | 0.58 | 5.96 | 1.59 |
| extract at 0.1 mg/mL | | | | |
| Comparative ethyl acetate | 2.92 | 0.4 | 8.21 | 0.58 |
| extract, decolorized with | | | | |
| Diaion, at 0.1 mg/mL | | | | |
| Comparative ethanolic extract | 4.56 | 1.1 | 6.77 | 1.65 |
| at 0.1 mg/mL | | | | |
| Comparative ethanolic extract, | 1.58 | 1.22 | 1.74 | 0.92 |
| decolorized with Diaion, at | | | | |
| 0.1 mg/mL | | | | |
| Comparative aqueous extract | 0.41 | 1.21 | 0.44 | 0.57 |
| at 0.1 mg/mL | | | | |

The tested extracts were the hydro-alcoholic extract of above-ground parts of evening primrose according to the invention; the comparative aqueous extract; the comparative ethanolic extract (decolorized or not) and the comparative ethyl acetate extract (decolorized or not).

The results show that among the different types of tested extracts of above-ground parts of evening primrose, the comparative ethanolic extracts, comparative ethyl acetate extract and the hydro-alcoholic extract of the invention induce the expression of markers involved in the forming of the cornified layer and barrier function (CNFN, FLG2, LCE3D and SPRR1A). However, the fold change is much higher for the hydro-alcoholic extract of the invention. The aqueous extract is inactive.

1. A hydro-alcoholic extract of above-ground parts (i.e. aerial parts) of Oenothera biennis.

2. The extract according to claim 1, which is obtained by extracting the above-ground parts in a mixture of water and at least one alcohol.

3. The extract according to claim 1, wherein the above-ground parts are selected from among the flowers, leaves and/or stems of Oenothera biennis.

4. The extract according to claim 2, wherein the mixture of water and at least one alcohol is a mixture comprising from 20 to 60 volume % of water relative to the total volume of the mixture, and from 40 to 80 volume % of alcohol relative to the total volume of the mixture.

5. The extract according to claim 2, wherein the mixture of water and at least one alcohol is a mixture comprising 50 volume % of water relative to the total volume of the mixture, and 50 volume % of alcohol relative to the total volume of the mixture.

6. The extract according to claim 1, which is obtained from above-ground parts, previously ground, and extracted with a hydro-alcoholic mixture.

7. The extract according to claim 1, which is obtained with a method comprising the following steps:
a): grinding into fine particles the dried, above-ground parts of evening primrose, in a grinder;
b): extracting, via maceration in a mixture of water and at least one alcohol, at a temperature of 15° C. to 45° C.; then
c): filtering the mixture obtained at b);
d): partly or fully evaporating the alcohol from the filtered mixture obtained at c); and
e): optionally freeze-drying the aqueous residue obtained at d).

8. The extract according to claim 1, which comprises at least two molecules selected from among oenothein B, quercetin, ellagic acid, hyperoside, isoquercitrin and luteoline-7-O-glucuronide.

9. The extract according to claim 1, which is a dry extract.

10. A composition comprising an extract according to claim 1 in a cosmetically acceptable medium.

11. A method for strengthening the barrier function and/or improving hydration of the skin, comprising applying an extract according to claim 1 onto skin.

12. A method for improving the quality of the skin and/or complexion; and/or for preventing and/or treating cosmetic signs associated with dry skin, comprising applying an extract according to claim 1 onto skin.

13. A non-therapeutic cosmetic method for care of keratin material, comprising the topical application to the keratin material of at least one extract according to claim 1, or of a composition comprising the at least one extract in a cosmetically acceptable medium.

14. The extract according to claim 2, wherein the at least one alcohol is a monoalcohol having 2 to 6 carbon atoms.

15. The extract according to claim 2, wherein the at least one alcohol is ethanol.

16. The extract according to claim 2, wherein the above-ground parts are selected from among the flowers, leaves and/or stems of Oenothera biennis.

17. The extract according to claim 3, wherein the mixture of water and at least one alcohol is a mixture comprising from 20 to 60 volume % of water relative to the total volume of the mixture, and from 40 to 80 volume % of alcohol relative to the total volume of the mixture.

18. The extract according to claim 3, wherein the mixture of water and at least one alcohol is a mixture comprising 50 volume % of water relative to the total volume of the mixture, and 50 volume % of alcohol relative to the total volume of the mixture.

19. The extract according to claim 2, which is obtained from above-ground parts, previously ground, and extracted with a hydro-alcoholic mixture.

20. The extract according to claim 3, which is obtained from above-ground parts, previously ground, and extracted with a hydro-alcoholic mixture
